(19) United States
(12) Patent Application Publication
(10) Pub. No.: US 2022/0319670 A1
(43) Pub. Date: Oct. 6, 2022

(54) Non-face-to-face AI occupational therapy assistance system and operating method using blockchain

(71) Applicant: INTHETECH, Daegu (KR)

(72) Inventors: Seung Ho CHOUN, Daegu (KR); Do Hyun AHN, Daegu (KR)

(21) Appl. No.: 17/599,898

(22) PCT Filed: Jul. 16, 2021
(86) PCT No.: PCT/KR2021/009188

§ 371 Date: Sep. 29, 2021

(30) Foreign Application Priority Data
Jul. 31, 2020 (KR) 10-2020-0095802

(51) Int. Cl. G16H 20/70; G16H 40/67; G06Q 20/38

(52) U.S. Cl. CPC G16H 20/70; G16H 40/67; G06Q 20/389

(57) ABSTRACT

Non-face-to-face AI occupational therapy assistance system and operating method using blockchain disclosed. The system includes a patient terminal configured for providing a cognitive ability evaluation tool and a cognitive rehabilitation training tool to a patient requiring training, a therapist terminal operated by an external occupational therapist; and an occupational therapy server configured for determining a degree of disability based on non-face-to-face evaluation of the patient's cognitive ability connected through the patient terminal, and according to the degree of disability, linking an AI occupational therapist or the external occupational therapist non-face-to-face through the therapist terminal to conduct the cognitive rehabilitation training.

### FIELD OF INVENTION

The present invention relates to an occupational therapy assistance system, and more particularly, to a non-face-to-face AI occupational therapy assistance system using blockchain and a method of operating a non-face-to-face AI occupational therapy assistance platform using blockchain.

### BACKGROUND

Cognitive and perceptual functions are the ability to accept various stimuli, judge and decide situations, and adapt to one's environment. These are essential elements for human beings to live as an independent individual. Therefore, it is difficult for persons with disabilities with impairments in cognitive and perceptual functions to independently perform daily activities and participate in social life normally.

Cognitive rehabilitation is a rehabilitation therapy that helps people with disabilities (brain damage, stroke, dementia, schizophrenia, cerebral palsy, chromosomal abnormality, etc.) to recover or compensate for cognitive and perceptual functions.

In the case of dementia, the cost of managing and treating dementia patients in South Korea alone amounts to 14.6 trillion KRW, and the number of dementia patients in South Korea has exceeded 750,000 and 1 in 10 people over the age of 65 is developing the disease.

The number of people diagnosed with ‘mild cognitive impairment’, a precursor to dementia, is rapidly increasing. 80% of the patients diagnosed with the mild cognitive impairment develop into dementia within 5 years, but the early treatment can reduce onset.

Currently, anti-dementia drug therapy has the highest therapeutic effect as a treatment for mild cognitive impairment, and various clinical trials such as drug therapy to activate brain function and anti-amyloid drug are in progress, but it has not shown a clear solution due to fatal side effects.

And in the conventional technologies, since the cognitive rehabilitation training contents for mild cognitive impairment or dementia are applied without distinction, the training efficiency is low and as a result, the improvement effect is low.

### SUMMARY OF INVENTION

### Technical Objectives

The present invention is to provide a non-face-to-face AI (Artificial Intelligence) occupational therapy assistance system using blockchain and a method of operating non-face-to-face AI occupational therapy assistance platform that may enhance the effectiveness of occupational therapy and provide systematic management and training by automatically performing evaluation and training of cognitive rehabilitation-related patients.

The present invention is to provide a non-face-to-face AI occupational therapy assistance system using blockchain and a method of operating non-face-to-face AI occupational therapy assistance platform that may provide the occupational therapy to more patients by enabling non-face-to-face cognitive rehabilitation therapy at a dementia center or at home even if an occupational therapist is not present and by enabling occupational therapy through AI according to the assessed level of cognitive ability.

The present invention is to provide a non-face-to-face AI occupational therapy assistance system using blockchain and a method of operating non-face-to-face AI occupational therapy assistance platform that may make any kinds of tampering impossible by encrypting and transmitting sensitive information such as evaluation of cognitive ability, cognitive rehabilitation training and feedback based on blockchain, and provide appropriate compensation to occupational therapists, experts, and patients according to their level of participation

Other objectives and advantages will be easily understood from the following description.

### Technical Solutions

According to one aspect of the present invention, a non-face-to-face artificial intelligence (AI) occupational therapy assistance system for supporting occupational therapy for cognitive rehabilitation is provided. The system includes a patient terminal configured for providing a cognitive ability evaluation tool and a cognitive rehabilitation training tool to a patient requiring training, a therapist terminal operated by an external occupational therapist; and an occupational therapy server configured for determining a degree of disability based on non-face-to-face evaluation of the patient's cognitive ability connected through the patient terminal, and according to the degree of disability, linking an AI occupational therapist or the external occupational therapist non-face-to-face through the therapist terminal to conduct the cognitive rehabilitation training.

The occupational therapy server includes a cognitive ability evaluation unit configured for evaluating the patient's cognitive ability based on the patient's evaluation result through the cognitive ability evaluation tool and selectively links the AI occupational therapist or the external occupational therapist and an AI occupational therapist unit, being activated when the degree of disability belongs to a mild cognitive impairment group and configured for performing an occupational therapy based on artificial intelligence, wherein the AI occupational therapist unit organizes a training content by automatically allocating training fields including memory, attention, and visual perception.

The AI occupational therapist unit counts scores for each field in N^{th} session of training, arranges each field in an order of scores, sums scores to estimate a share of each field, and organizes the training content so that the field with a relatively high share in N^{th} session of training has a relatively low allocation ratio in (N+1)^{th} session of training by matching share of each field in a reverse order.

The system further includes an expert terminal being operated by an expert on cognitive rehabilitation training, wherein the occupational therapy server further includes a training content market operation unit configured for registering a new training content received through the expert terminal or the therapist terminal and providing the new training content for use in the occupational therapy, wherein the new training content is automatically applied to the AI occupational therapist unit and the therapist terminal cites the new training content through search.

The system further includes a therapist evaluation unit configured for dividing the evaluation result for the occupational therapist linked from the patient into blocks to distributively store in a blockchain network, and generating and transferring a reward currency to the occupational therapist exceeding a preset minimum requirement according to the evaluation result and the patient performing the evaluation.

The therapist evaluation unit displays an evaluation request screen related to occupational therapy only in the patient terminal at a random time during a session of training in progress through screen sharing in connection with the patient terminal and the therapist terminal.

Other aspects, features, and advantages will be more apparent from accompanying drawings, claims and detailed description.

### Effects of Invention

According to embodiment of the present invention, it is advantageous that the effectiveness of occupational therapy can be increased by automatically performing evaluation and training of cognitive rehabilitation-related patients, and systematic management and training can be provided.

It is also advantageous that non-face-to-face cognitive rehabilitation therapy is possible at the dementia center or at home even though an occupational therapist is not present and occupational therapy through AI becomes possible according to the assessed level of cognitive ability to provide occupational therapy to more patients.

It is also advantageous that any kinds of tampering become impossible by encrypting and transmitting sensitive information such as evaluation of cognitive ability, cognitive rehabilitation training and feedback based on blockchain and appropriate reward can be provided to occupational therapists, experts, patients, and so on according to the degree of participation.

### BRIEF DESCRIPTION OF ACCOMPANYING DRAWINGS

FIG. 1 is a block diagram of a non-face-to-face AI occupational therapy assistance system to which a block chain is applied according to one embodiment of the present invention;

FIG. 2 illustrates a cognitive ability evaluation and training for the patient;

FIG. 3 illustrates a training allocation according to the cognitive ability evaluation result;

FIG. 4 illustrates a content market in operation;

FIG. 5 illustrates a training content planning and registration by an expert;

FIG. 6 is a block diagram of a program executed in a patient terminal of a non-face-to-face AI occupational therapy assistance system to which a block chain is applied according to one embodiment of the present invention;

FIG. 7 is a diagram of information update and block generation in the evaluation tool;

FIG. 8 illustrates graphs of training results by field; and

FIG. 9 is a diagram showing a flow for training planning and reward.

### DETAILED DESCRIPTION

The invention can be modified in various forms and specific embodiments will be described and shown below. However, the embodiments are not intended to limit the invention, but it should be understood that the invention includes all the modifications, equivalents, and replacements belonging to the concept and the technical scope of the invention.

If it is mentioned that an element is “connected to” or “coupled to” another element, it should be understood that still another element may be interposed therebetween, as well as that the element may be connected or coupled directly to another element. On the contrary, if it is mentioned that an element is “connected directly to” or “coupled directly to” another element, it should be understood that still another element is not interposed therebetween.

Terms such as first, second, etc., may be used to refer to various elements, but, these element should not be limited due to these terms. These terms will be used to distinguish one element from another element.

The terms used in the following description are intended to merely describe specific embodiments, but not intended to limit the invention. An expression of the singular number includes an expression of the plural number, so long as it is clearly read differently. The terms such as “include” and “have” are intended to indicate that features, numbers, steps, operations, elements, components, or combinations thereof used in the following description exist and it should thus be understood that the possibility of existence or addition of one or more other different features, numbers, steps, operations, elements, components, or combinations thereof is not excluded.

Elements of an embodiment described below with reference to the accompanying drawings are not limited to the corresponding embodiment, may be included in another embodiment without departing from the technical spirit of the invention. Although particular description is not made, plural embodiments may be embodied as one embodiment.

In describing the invention with reference to the accompanying drawings, like elements are referenced by like reference numerals or signs regardless of the drawing numbers and description thereof is not repeated. If it is determined that detailed description of known techniques involved in the invention makes the gist of the invention obscure, the detailed description thereof will not be made.

Terms such as ˜part, ˜unit, ˜module mean an element configured for performing a function or an operation. This can be implemented in hardware, software or combination thereof.

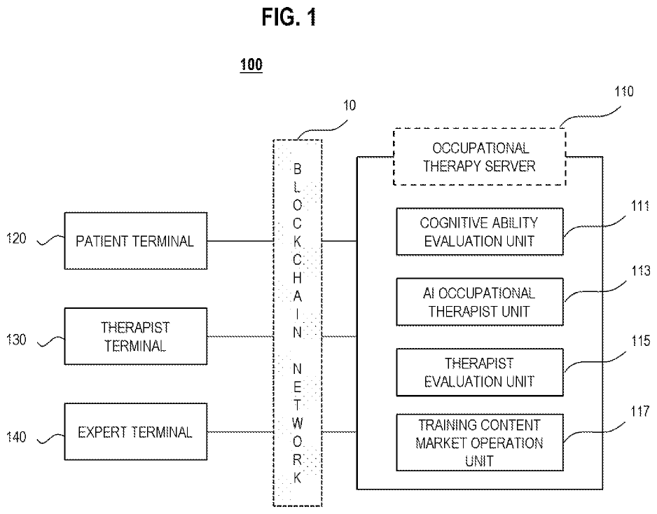

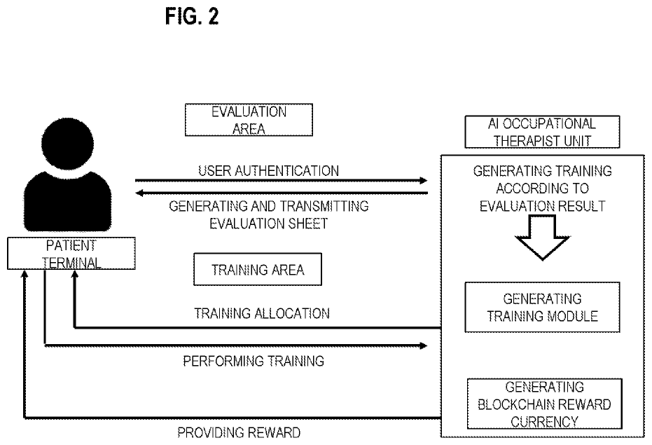

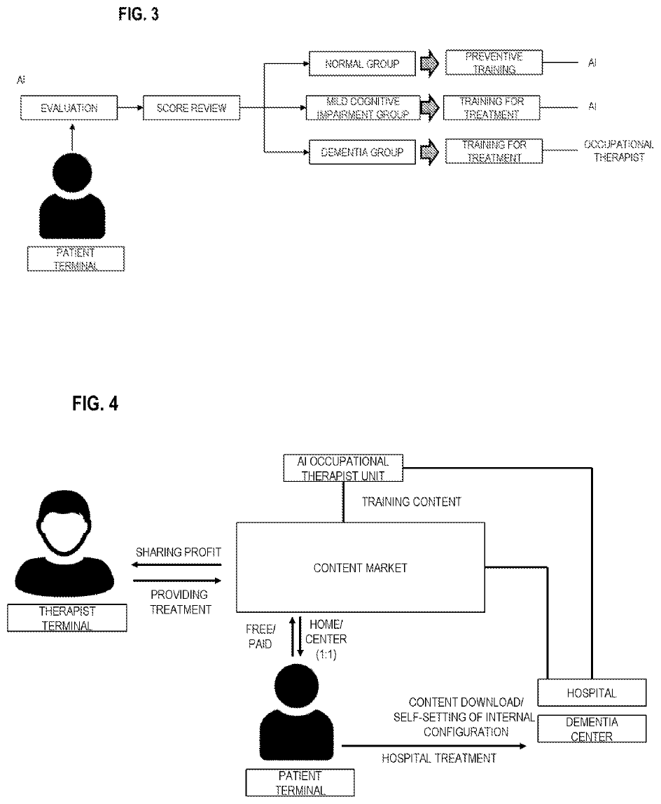

FIG. 1 is a block diagram of a non-face-to-face AI occupational therapy assistance system to which a block chain is applied according to one embodiment of the present invention, FIG. 2 illustrates a cognitive ability evaluation and training for the patient, FIG. 3 illustrates a training allocation according to the cognitive ability evaluation result, FIG. 4 illustrates a content market in operation, and FIG. 5 illustrates a training content planning and registration by an expert.

The non-face-to-face AI occupational therapy assistance system and the method of operating a non-face-to-face AI occupational therapy assistance platform using blockchain are characterized by enabling non-face-to-face cognitive rehabilitation therapy at a dementia center or at home even though the occupational therapist is not in the same space with the patient, and occupational therapy through AI according to the assessed level of cognitive ability to provide occupational therapy to more patients.

The non-face-to-face AI occupational therapy assistance system 100 using blockchain according to one embodiment includes an occupational therapy server 110, a patient terminal 120, a therapist terminal 130, and an expert terminal 140. The occupational therapy server 110 may function as a non-face-to-face AI occupational therapy assistance platform.

The patient terminal 120 is a terminal device used by a patient subject to occupational therapy, that is, a patient requiring cognitive rehabilitation training, a patient with dementia or mild cognitive impairment, or a user requiring prevention. The terminal device is a computing device on which a patient application for occupational therapy is loaded and executed, and may be, for example, one of a smartphone, a tablet computer, a desktop computer, a laptop computer, and the like.

The therapist terminal 130 is a terminal device used by occupational therapists who engage in occupational therapy. The terminal device is a computing device on which an occupational therapy application for occupational therapy is loaded and executed, and may be, for example, one of a smartphone, a tablet computer, a desktop computer, a laptop computer, and the like.

The expert terminal 140 is a terminal device used by experts for cognitive rehabilitation training. The terminal device is a computing device that can be executed by mounting a professional application for occupational therapy, and may be, for example, one of a smartphone, a tablet computer, a desktop computer, a laptop computer, and the like.

The occupational therapy server 110 communicates with the patient terminal 120, the therapist terminal 130, and the expert terminal 140, and automatically evaluates cognitive abilities based on AI for the authenticated patient. According to the evaluated level of cognitive ability, the occupational therapy server 110 divides cognitive rehabilitation training for patients into an AI occupational therapist that automatically performs according to a predetermined scenario and an external occupational therapist who performs professional cognitive rehabilitation training based on experience, and have occupational therapy be performed.

In addition, the content for cognitive rehabilitation training for occupational therapy can be uploaded to the market through the therapist terminal 130 or the expert terminal 140 so that other occupational therapists or other experts can utilize it, and rewards can be provided according to the degree of use.

In addition, during non-face-to-face cognitive rehabilitation training, the patient can conduct an evaluation of the occupational therapist in charge without the occupational therapist being aware of it, and provide bonuses for the occupational therapists who obtains positive evaluations according to the results.

By interposing the blockchain network 10 between the occupational therapy server 110 and each of the terminals (patient terminal 120, therapist terminal 130, expert terminal 140) and communicating data (e.g., patient basic information, training records, training results, etc.) that is encrypted in blocks and recorded in each node of the blockchain network 10, data may not be tampered, at the same time, may be accessible only by those who are permitted to access the data, and it may be possible to make it impossible to check the records for abnormal access.

The occupational therapy server 110 includes a cognitive ability evaluation unit 111 and the AI occupational therapist unit 113.

Referring to FIG. 2, the cognitive ability evaluation unit 111 evaluates the cognitive ability of a patient authenticated through the patient terminal 120.

User authentication of the patient through the patient terminal 120 may be performed through input of a pre-registered ID and password, or biometric recognition (fingerprint recognition, face recognition, etc.).

When the authenticated patient accesses through the patient terminal 120, the cognitive ability evaluation is performed in a non-face-to-face manner. Cognitive ability evaluation is performed online, and an evaluation sheet for cognitive ability evaluation may be generated and transmitted to the patient terminal 120.

Also, the cognitive ability evaluation may be performed using audio and/or video. In this case, the patient terminal 120 needs to be equipped with a multimedia device such as a display and a camera for an evaluation using video, and a speaker and a microphone for an evaluation using audio.

In the course of the evaluation in the cognitive ability evaluation unit 111, AI evaluation based on artificial intelligence may be performed, and the cognitive ability may be scored in a preset manner.

Cognitive ability evaluation unit 111 may determine a subject in charge of cognitive rehabilitation training of the patient according to the evaluation score.

Referring to FIG. 3, the cognitive ability evaluation score may be largely divided into three groups, such as a normal group, a mild cognitive impairment group, and a dementia group. A preventive training among cognitive rehabilitation trainings may be performed for patients in the normal group, and a training for treatment may be performed for patients in the mild cognitive impairment group, both may be performed by the AI occupational therapist. And the training for treatment may be performed for patients in the dementia group by linking a professional external occupational therapist.

The cognitive ability evaluation unit 111 activates the AI occupational therapist unit 113 for the patient in the normal group or the mild cognitive impairment group to use the training contents of which share is distributed for each detailed field according to a preset algorithm, thereby reducing the manpower required for occupational therapy by enabling automated training to be performed according to a training schedule specifically designed for the patient.

In addition, the cognitive ability evaluation unit 111 links the patient in the dementia group with the therapist terminal 130 and links the patient and the external occupational therapist one-on-one so that the cognitive rehabilitation training may be performed through professional occupational therapy.

Referring back to FIG. 2, the AI occupational therapist unit 113 generates a training module by applying automated computerized cognitive rehabilitation training content to patients who are classified to be trained by the AI occupational therapist (normal group, mild cognitive impairment group) as a result of evaluation by the cognitive ability evaluation unit 111. The AI occupational therapist unit 113 transmits the generated training module to the patient terminal 120 to allocate appropriate training to the patient and perform the training.

The AI occupational therapist unit 113 may automatically set a difficulty level of the next training for the patient according to the training result. For example, if the training result is excellent, the cognitive rehabilitation can be sped up through relatively high level training, and in the opposite case, the cognitive rehabilitation rate can be slowed down through relatively low difficulty training.

By transmitting the training results during the training performed by the AI occupational therapist unit 113 to the expert terminal 140, the expert may intervene and provide feedback. This enables an expert to review and coach the individual characteristics of each patient in the same group according to the cognitive ability evaluation, so that occupational therapy suitable for the patient can be made.

In addition, the AI occupational therapist unit 113 may generate reward currency for training performance by registering in the blockchain network 10 in relation to the produced training module. If the patient performs above a preset minimum requirement in relation to the training module assigned to the patient terminal 120 when judging from the training results transmitted from the patient terminal 120, the reward currency generated above may be transferred to the corresponding patient terminal 120 to provide reward.

When the therapist terminal 130 is connected by the cognitive ability evaluation unit 111 for patient in the dementia group, the external occupational therapist may perform occupational therapy while communicating with the patient in the non-face-to-face manner.

In this case, the external occupational therapist can directly select the training method suitable for the patient's situation. In addition, it is possible to directly guide the patient's training through audio and video. Training results may be analyzed, and if necessary, a visit can be requested. When there is a request for the visit, a reservation may be automatically made in connection with a hospital reservation system (not shown).

In addition, the occupational therapy server 110 may further include a training content market operation unit 117.

The training content market operation unit 117 manages a database for storing various contents for cognitive rehabilitation training. In addition, the training content market operation unit 117 registers training content to be used during the occupational therapy, and operates the training content market that allows AI occupational therapists 113 and therapist terminals 130 to search and cite training content to perform the occupational therapy.

Training content may be registered in the training content market through the expert terminal 140 by an expert on cognitive rehabilitation training. In this case, a registration process of the training content may be provided in a predetermined format through an application executed in the expert terminal 140. Alternatively, the training content may be registered in the market through the therapist terminal 130 by the occupational therapist.

Referring to FIG. 5, the expert may register a plan for training online through the expert terminal 140. The registered plan can be accessed and checked by a content development company (developer), and content can be produced according to the plan. When the produced content is confirmed and finalized through expert online feedback, the expert terminal 140 transmits a content registration request to the training content market operation unit 117 to upload new training content to the training content market.

The uploaded new training content is automatically applied to AI occupational therapists and can be cited as needed during occupational therapy, and in the case of external occupational therapists, it can be used for training through direct selection and citation after searching.

The training content market operation unit 117 may give a ranking to the training content registered in the training content market. Content with a high number of citations by the AI occupational therapist unit 113 and the therapist terminal 130 is given a relatively high score and priority, so that more citations can be made by placing it in a higher rank when searching.

In addition, for experts who have registered training content having a high ranking, reward for content registration may be performed by transferring reward currency through the blockchain network 10 as described above.

In addition, the occupational therapy server 110 may further include a therapist evaluation unit 115.

According to the evaluation result by the cognitive ability evaluation unit 111, each patient is assigned an AI occupational therapist or an external occupational therapist according to the degree of cognitive impairment. The occupational therapy can be performed through video and audio in the non-face-to-face manner.

The external occupational therapist can be connected to the patient terminal 120 through the therapist terminal 130 via network, and if necessary, the patient's training process can be monitored while sharing the screen of the patient terminal 120.

The patient terminal 120 may randomly display an evaluation request screen regarding the current occupational therapy while training according to occupational therapy is in progress. This evaluation request screen is not displayed on the therapist terminal 130 that is sharing the screen of the patient terminal, so the external occupational therapist cannot recognize whether there is the evaluation request.

The evaluation result regarding occupational therapy by the patient is divided into blocks through the blockchain network 10 and distributively stored in each node to make the tampering of the evaluation result impossible. For patients participating in this evaluation, a predetermined reward can be made by generating and transferring the reward currency.

The therapist evaluation unit 115 may randomly transmit the evaluation request screen to the patient terminal 120 currently undergoing occupational therapy and collect evaluation results accordingly. For external occupational therapists whose collected evaluation results exceed a preset minimum requirement, reward currency that proves the superiority of treatment can be generated and transferred to the therapist terminal 130 so that additional reward for occupational therapy can be made to induce more active occupational therapy.

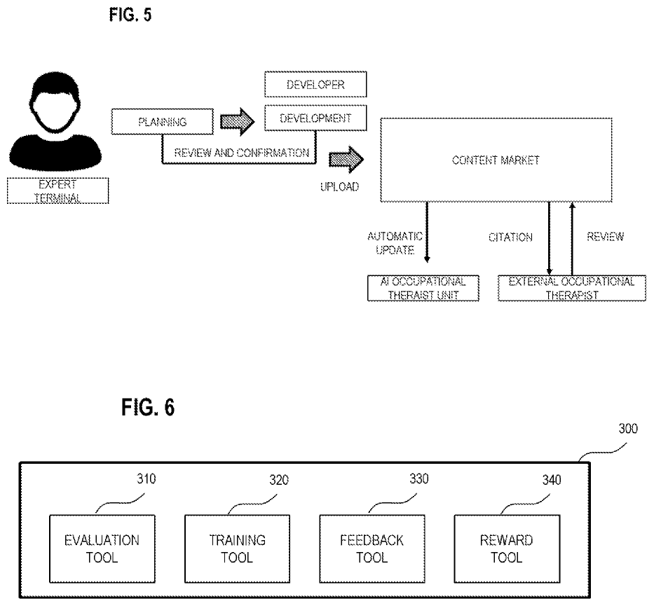

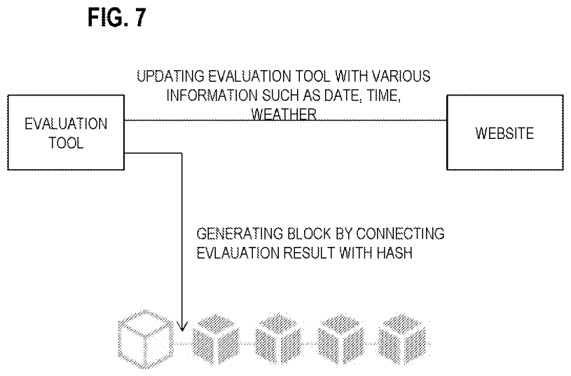

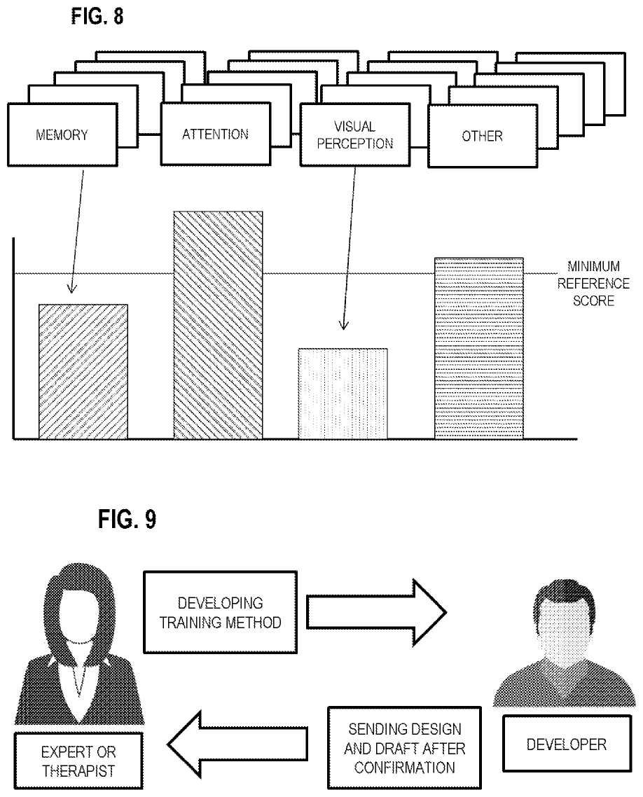

FIG. 6 is a block diagram of a program executed in a patient terminal of a non-face-to-face AI occupational therapy assistance system to which a block chain is applied according to one embodiment of the present invention, FIG. 7 is a diagram of information update and block generation in the evaluation tool, and FIG. 8 illustrates graphs of training results by field.

The patient terminal 120 is a computing device equipped with a graphical user interface (GUI). Each patient may be given an individual ID capable of logging in. If the patient can use the terminal, there is no need for a guardian, and if the patient cannot freely use the terminal, the guardian may operate the patient's terminal instead. In this specification, a patient or a guardian may be collectively referred to as a user.

When a user logs in, a program (application) capable of cognitive ability evaluation and cognitive rehabilitation training is displayed on the terminal screen.

When logging in, as shown in FIG. 6, the user can face a program 300 including an evaluation tool 310, a training tool 320, a feedback tool 330, and a reward tool 340.

The evaluation tool 310 may be composed of four evaluation fields of attention evaluation, visual perception evaluation, memory evaluation, and other evaluations.

The evaluation tool 310 brings basic information such as date, time, and weather information from an external website. In addition, the evaluation problem can be updated by gathering information about the user's usual surroundings, such as orientation, from an external website.

The evaluation consists of a number of problems, and by having the problems presented through a random function, the user can encounter a different problem each time.

When the user completes the evaluation, a block containing the evaluation results (evaluation details and evaluation score) for the user is generated (see FIG. 7). By connecting the evaluation results with a hash, a block can be created and registered in the blockchain network 10.

After the evaluation is completed, a secret key can be generated and signed, and then transmitted to the occupational therapy server 110.

The occupational therapy server 110, in particular, the cognitive ability evaluation unit 111 may receive the evaluation results, decrypt the results with the public key, and check the results. If a diagnosis is required after checking the evaluation score, an evaluation opinion may be generated and recorded, and transmitted to the expert terminal 140 so that the expert diagnosis can be made.

And the cognitive ability evaluation unit 111 configures training according to the evaluation results. The training configuration can be arbitrarily conducted by the external occupational therapist or can be classified based on AI so that the training configuration can be made automatically.

For example, referring to FIG. 8, since the memory field and the visual perception field were evaluated to be lower than the minimum reference score, the training content may be configured in a way that further strengthens the memory field and the visual perception field.

In addition, when the training is not completed in the first session but is configured in multiple sessions, the training content may be dynamically configured as follows.

In the first session of training, scores for each field (memory, attention, visual perception, etc.) are counted, and placement is carried out from the lowest score to the highest score. The four scores are summed, and the percentage of each score in the sum is estimated. It can be organized in such way that the field with the highest percentage receives the smallest percentage of problems in the next session of training.

TABLE 1

| | | | (N + 1)^{th} session |
|---|---|---|---|
| Field | Score in N^{th} session | Share(%) | allocation ratio(%) |
| Attention | 40 | 16 | 36 |
| Visual perception | 50 | 20 | 28 |
| Memory | 70 | 28 | 20 |
| Other | 90 | 36 | 16 |
| Sum | 250 | 100 | 100 |

Referring to the Table 1, the training score for the N^{th} session is a total of 250 points with 40 points for attention, 50 points for visual perception, 70 points for memory and 90 points for others. In this case, the share of each field is 16% for attention, 20% for visual perception, 28% for memory, and 36% for other.

In this case, in the (N+1)^{th} session of training, the training allocation ratio can be calculated by arranging the occupancy in the reverse order. That is, in the (N+1)th session of training, 36% of the attention field corresponding to the share of other field in the N^{th} session is allocated to the attention field, 28% corresponding to the share of the memory field in the N^{th} session is allocate to the visual perception field., 20% corresponding to the share of the visual perception field in the N^{th} session is allocated to the memory field, and 16% corresponding to the share of the attention field in the N^{th} session is allocated to other field, so that the total can be 100%.

Up to a predetermined numbers of session (e.g., 30 sessions), the next session of training may be allocated based on the grades for each round, and when it exceeds the predetermined numbers of session, the training may be allocated based on the cumulative average score.

When the occupational therapist (AI occupational therapist or external occupational therapist) records the training results, a transaction can be created and encrypted. These training results may be transmitted to users and experts through the blockchain network 10.

The expert terminal 140 may receive the training results and decrypt it with the public key to check the results. The training results and evaluations can be checked as a whole, and whether to improve can be determined.

Based on the determination, training instructions can be sent to the occupational therapist or the user can be requested to visit.

FIG. 9 is a diagram showing a flow for training planning and reward.

Referring to FIG. 9, in this embodiment, the therapist and/or the expert may prepare a training plan and send it to the developer.

The developer sends the draft content including the design that meets the original designer's intention to the therapist or expert, and obtains final confirmation.

The developed training content can be 2D or 3D, and it can be registered in the aforementioned training content market to be cited by other therapists (AI occupational therapist, external occupational therapist).

For training contents that have been cited, electronic money that can be automatically converted into cash can be transferred to the expert or therapist who planned it.

In addition, electronic money for internal use can be paid as a reward to users who have been diligently trained according to their performance. Electronic money for internal use can be used to pay for expenses incurred in the future occupational therapy process.

The above-described method of operating a non-face-to-face AI occupational therapy assistance platform using blockchain may also be implemented in the form of a non-transitory recording medium including instructions executable by a computer, such as an application or program module executed by a computer. Non-transitory computer-readable medium can be any available media that can be accessed by a computer and includes both volatile and nonvolatile medium, removable and non-removable media. In addition, non-transitory computer-readable medium may include computer storage medium. Computer storage medium includes both volatile and nonvolatile, removable and non-removable media implemented in any method or technology for storage of information such as computer readable instructions, data structures, program modules or other data.

The above-described method of operating a non-face-to-face AI occupational therapy assistance platform using blockchain may be executed by an application (which may include a program included in a platform or operating system by default installed in the terminal) installed by default in the terminal, and by an application (i.e., program) that a user manually installed in the terminal after downloading from an application store server, or an application providing server such as a web server related to the application or service. In this sense, the above-described work support method with screen sharing may be implemented as an application (i.e., program) installed by default in the terminal or manually installed by a user, and may be recorded in the non-transitory computer-readable recording medium such as the terminal.

While the invention has been described above with reference to exemplary embodiments, it will be understood by those skilled in the art that the invention can be modified and changed in various forms without departing from the concept and scope of the invention described in the appended claims.

What is claimed is:

1. A non-face-to-face artificial intelligence (AI) occupational therapy assistance system for supporting occupational therapy for cognitive rehabilitation, comprising:
a patient terminal configured for providing a cognitive ability evaluation tool and a cognitive rehabilitation training tool to a patient requiring training;
a therapist terminal operated by an external occupational therapist; and
an occupational therapy server configured for determining a degree of disability based on non-face-to-face evaluation of the patient's cognitive ability connected through the patient terminal, and according to the degree of disability, linking an AI occupational therapist or the external occupational therapist non-face-to-face through the therapist terminal to conduct the cognitive rehabilitation training.

2. The system according to claim 1, wherein the occupational therapy server comprises:
a cognitive ability evaluation unit configured for evaluating the patient's cognitive ability based on the patient's evaluation result through the cognitive ability evaluation tool and selectively links the AI occupational therapist or the external occupational therapist; and
an AI occupational therapist unit, being activated when the degree of disability belongs to a mild cognitive impairment group and configured for performing an occupational therapy based on artificial intelligence,
wherein the AI occupational therapist unit organizes a training content by automatically allocating training fields including memory, attention, and visual perception.

3. The system according to claim 2, wherein the AI occupational therapist unit counts scores for each field in N^{th} session of training, arranges each field in an order of scores, sums scores to estimate a share of each field, and organizes the training content so that the field with a relatively high share in N^{th} session of training has a relatively low allocation ratio in (N+1)^{th} session of training by matching share of each field in a reverse order.

4. The system according to claim 2 further comprising an expert terminal being operated by an expert on cognitive rehabilitation training,
wherein the occupational therapy server further includes a training content market operation unit configured for registering a new training content received through the expert terminal or the therapist terminal and providing the new training content for use in the occupational therapy,
wherein the new training content is automatically applied to the AI occupational therapist unit and the therapist terminal cites the new training content through search.

5. The system according to claim 2 further comprising a therapist evaluation unit configured for dividing the evaluation result for the occupational therapist linked from the patient into blocks to distributively store in a blockchain network, and generating and transferring a reward currency to the occupational therapist exceeding a preset minimum requirement according to the evaluation result and the patient performing the evaluation.

6. The system according to claim 5, wherein the therapist evaluation unit displays an evaluation request screen related to occupational therapy only in the patient terminal at a random time during a session of training in progress through screen sharing in connection with the patient terminal and the therapist terminal.
